(19) United States
(12) Patent Application Publication
(10) Pub. No.: US 2019/0060365 A1
(43) Pub. Date: Feb. 28, 2019

(54) PHARMACEUTICAL COMPOSITION FOR TREATING CHRONIC OBSTRUCTIVE PULMONARY DISEASE AND METHOD THEREOF

(71) Applicant: Meridigen Biotech Co., Ltd., Taipei City (TW)

(72) Inventors: Chang-Yo HSUAN, Taipei City (TW); Willie LIN, Taipei City (TW); Yu-Chin SU, Taipei City (TW); Tang-bo Chung WU, Taipei City (TW); Yu-Chieh Wen, Taipei City (TW)

(73) Assignee: Meridigen Biotech Co., Ltd., Taipei City (TW)

(21) Appl. No.: 15/686,464

(22) Filed: Aug. 25, 2017

(51) Int. Cl. A61K 35/28; A61K 47/42

(52) U.S. Cl. CPC A61K 35/28; A61K 47/42

(57) ABSTRACT

Disclosed is a pharmaceutical composition for treating chronic obstructive pulmonary disease, comprising an effective amount of human mesenchymal stem cells, human serum albumin, and a pharmaceutically acceptable carrier or diluent. Also disclosed is a method for treating chronic obstructive pulmonary disease in a subject in need thereof, the method comprising administering to the subject a pharmaceutical composition comprising an effective amount of human mesenchymal stem cells, human serum albumin, and a pharmaceutically acceptable carrier or diluent.

### FIELD OF THE INVENTION

The present invention pertains to a pharmaceutical composition for treating chronic obstructive pulmonary disease, as well as a method thereof.

### BACKGROUND OF THE INVENTION

Stem cells are multi-potent cells with a wide range of potential therapeutic applications [1]. Stem cell therapy has been a promising therapy for pulmonary diseases, including asthma, bronchopulmonary dysplasia (BPD), and chronic obstructive pulmonary disease (COPD) [2-4]. In preclinical studies, stem cells treatment has shown promising result against lung disorders [5-7]. Furthermore, clinical studies have demonstrated that the administration of stem cells to patients having advanced-stage COPD is safe and without significant adverse effects [8-11]. In stem cell-based therapies, intravenous infusion is commonly used to deliver stem cells for preclinical and clinical applications in pulmonary diseases [12-15].

U.S. Pat. No. 9,415,036 B2 discloses a pharmaceutical composition for the acute and/or chronic treatment or prevention of osteoarticular diseases including an adequate pharmaceutical carrier or diluent, a polysaccharide and/or a glycosaminoglycan, an anti-inflammatory agent and stem cells.

### BRIEF SUMMARY OF THE INVENTION

In one aspect, the present invention provides a pharmaceutical composition for treating chronic obstructive pulmonary disease. The pharmaceutical composition includes an effective amount of human mesenchymal stem cells, human serum albumin, and a pharmaceutically acceptable carrier or diluent.

In another aspect, the present invention provides a method for treating chronic obstructive pulmonary disease in a subject in need thereof. The method comprises the step of administering to the subject a pharmaceutical composition comprising an effective amount of human mesenchymal stem cells, human serum albumin, and a pharmaceutically acceptable carrier or diluent.

According to the present invention, the pharmaceutical composition may be prepared by a method comprising: mixing the human mesenchymal stem cells with the pharmaceutically acceptable carrier or diluent, which is supplemented with an effective amount of human serum albumin.

It is to be understood that both the foregoing general description and the following detailed description are exemplary and explanatory only and are not restrictive of the invention.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred.

In the drawings:

FIG. 1A shows that HSA has no effects on viability of mesenchymal stem cells; and FIG. 1B shows that HSA has no effects on cell number of mesenchymal stem cells.

FIG. 2 shows that HSA enhances the efficacy of stem cell therapy for chronic obstructive pulmonary disease. *P<0.05, **P<0.01 and ***P<0.001, as compared to Group 2. #P<0.05, ##P<0.01 and ###P<0.001, as compared to Group 4.

### DETAILED DESCRIPTION OF THE INVENTION

In one aspect, the present invention provides a pharmaceutical composition for treating chronic obstructive pulmonary disease (COPD). The pharmaceutical composition includes an effective amount of human mesenchymal stem cells, human serum albumin (HSA), and a pharmaceutically acceptable carrier or diluent.

In another aspect, the present invention provides a method for treating chronic obstructive pulmonary disease (COPD) in a subject in need thereof. The method comprises the step of administering to the subject a pharmaceutical composition comprising an effective amount of human mesenchymal stem cells, human serum albumin (HSA), and a pharmaceutically acceptable carrier or diluent.

The term “mesenchymal stem cells” as used herein includes cells isolated from tissues of adults, such as bone marrow, a fat cell, and a periodontal membrane, as well as cells isolated from tissues of fetus, placenta, and cord blood. In some examples of the present invention, the mesenchymal stem cells are derived from a placenta-related tissue selected from the group consisting of amniotic membrane, chorionic disk, chorionic membrane, and umbilical cord.

According to one embodiment, the pharmaceutically acceptable carrier or diluent is a normal saline.

According to the present invention, the amount of human serum albumin is effective in enhancing the efficacy of stem cell therapy for COPD, and can be determined by a person of ordinary skill in the art through routine experimentation.

According to certain embodiments of the present invention, the amount of the human serum albumin is ranging from 0.5% (w/v) to 25% (w/v), preferably 1% (w/v) to 10% (w/v), based on the volume of the pharmaceutically acceptable carrier or diluent.

As used herein, w/v means g/mL.

According to the present invention, the pharmaceutical composition may be prepared by a process comprising: mixing the human mesenchymal stem cells with the pharmaceutically acceptable carrier or diluent, which is supplemented with the human serum albumin.

Said process may further comprise incubating the human mesenchymal stem cells, as a suspension culture, in the pharmaceutically acceptable carrier or diluent, for a period of time to transform the condition of the human mesenchymal stem cells to one which is more effective in treating COPD in a stem cell-based therapy. A person of ordinary skill in the art may determine, through routine experimentation, an adequate period of time for incubating the human mesenchymal stem cells, such that the pharmaceutical composition is more effective in treating chronic obstructive pulmonary disease as compared to a pharmaceutical composition which is not “activated” by said process before use. In other words, the method for treating chronic obstructive pulmonary disease of the present invention may further comprise the preliminary steps of: mixing the human mesenchymal stem cells with the pharmaceutically acceptable carrier or diluent, which is supplemented with the human serum albumin, and incubating the pharmaceutical composition to transform the condition of the human mesenchymal stem cells to one which is more effective in treating COPD in a stem cell-based therapy.

However, since a pharmaceutical composition comprising stem cells is generally administered to a subject in need thereof gradually over a relatively long period of time, the pharmaceutical composition of the present invention may also be used shortly after the human mesenchymal stem cells are mixed with the pharmaceutically acceptable carrier or diluent supplemented with human serum albumin.

The present invention is further illustrated by the following examples, which are provided for the purpose of demonstration rather than limitation.

### EXAMPLES

### Example 1

### Preparation of Mesenchymal Stem Cells

Full-term placentas were collected after obtaining written informed consent from donors. MSCs were derived from amniotic membrane (AM), chorionic disk (CD), chorionic membrane (CM), and umbilical cord (UC). Placenta-derived mesenchymal stem cells were cultured, expanded and maintained in α-MEM with FBS and basic FGF at 37° C., saturating humidity and 5% CO₂, and were sub-cultured when cells reached 80% confluence. The cells are stored under low temperature conditions before use.

### Example 2

### HSA has no Effects on Viability and Cell Number of Mesenchymal Stem Cells

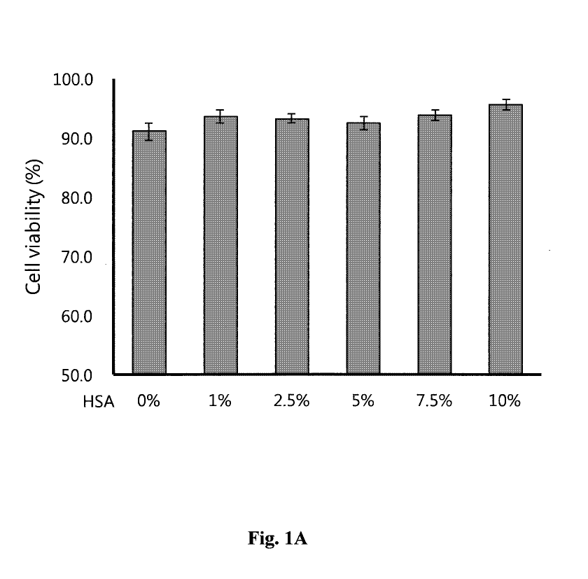

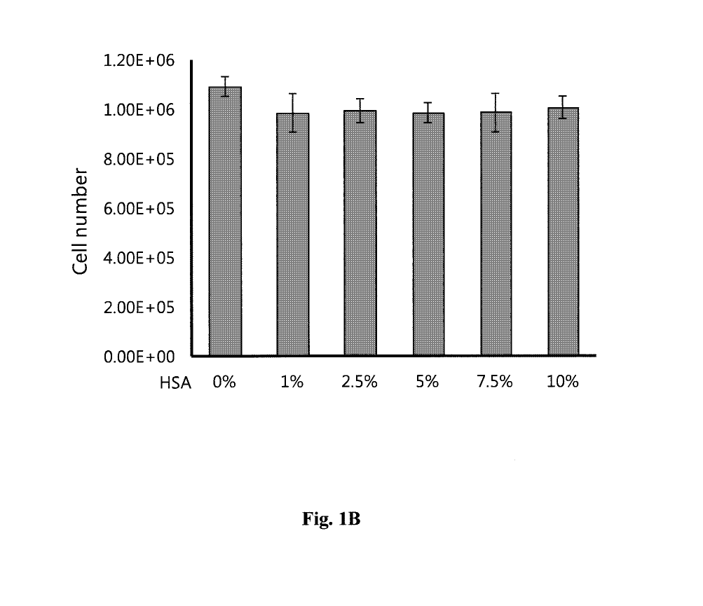

1×10⁷ mesenchymal stem cells (prepared as described in Example 1) were mixed with normal saline supplemented with different amounts (0, 1% (w/v), 2.5% (w/v), 5% (w/v), 7.5% (w/v), and 10% (w/v), based on the volume of normal saline) of human serum albumin (HAS), and incubated for 4 hours. Cell viability and cell number were measured by a cell counter (NucleoCounter® NC-250, ChemoMetec). The results are shown in FIG. 1A and FIG. 1B.

### Example 3

### HSA Enhances the Efficacy of Stem Cell Therapy for Chronic Obstructive Pulmonary Disease

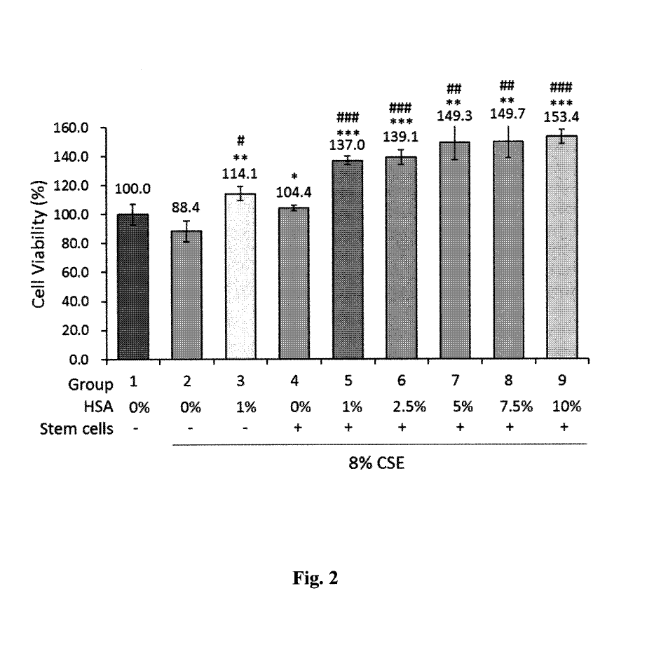

1×10⁷ mesenchymal stem cells (prepared as described in Example 1) were mixed with normal saline supplemented with different amounts (0, 1% (w/v), 2.5% (w/v), 5% (w/v), 7.5% (w/v), and 10% (w/v), based on the volume of normal saline) of HSA, and incubated for 4 hours. Human normal lung fibroblasts MRC-5, were treated with 8% cigarette smoke extract (CSE) for 24 hours, and then co-cultured with or without the mesenchymal stem cells for 48 hours. The mesenchymal stem cells were co-cultured with the CSE-damaged MRC-5 cells by using 24-well insert plates with 0.4 μm membrane pore sizes. Viability of the MRC-5 cells was analyzed by CCK-8 assay. The results are shown in FIG. 2. As can be seen in FIG. 2, HSA (1% (w/v)) or mesenchymal stem cells alone exhibited moderate beneficial effects on the viability of CSE-damaged MRC-5 cells; however, the combination of HSA and mesenchymal stem cells showed unexpected synergistic effects in recovering viability of CSE-damaged MRC-5 cells.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

### REFERENCES

[1] Spees, J. L., R. H. Lee, and C. A. Gregory, Mechanisms of mesenchymal stem/stromal cell function. Stem Cell Res Ther, 2016. 7(1): p. 125.
[2] Jin, Z., et al., Biological effects and mechanisms of action of mesenchymal stem cell therapy in chronic obstructive pulmonary disease. J Int Med Res, 2015. 43(3): p. 303-10.
[3] Mohammadian, M., et al., Effect of bone marrow derived mesenchymal stem cells on lung pathology and inflammation in ovalbumin-induced asthma in mouse. Iranian Journal of Basic Medical Sciences, 2016. 19(1): p. 55-63.
[4] Mobius, M. A. and B. Thebaud, Cell Therapy for Bronchopulmonary Dysplasia: Promises and Perils. Paediatr Respir Rev, 2016. 20: p. 33-41.
[5] Huh, J. W., et al., Bone marrow cells repair cigarette smoke-induced emphysema in rats. Am J Physiol Lung Cell Mol Physiol, 2011. 301.
[6] Hoffman, A. M., et al., Lung-derived mesenchymal stromal cell post-transplantation survival, persistence, paracrine expression, and repair of elastase-injured lung. Stem Cells Dev, 2011. 20.
[7] Schweitzer, K. S., et al., Adipose stem cell treatment in mice attenuates lung and systemic injury induced by cigarette smoking. Am J Respir Crit Care Med, 2011. 183.
[8] Weiss, D. J., et al., A Placebo-Controlled, Randomized Trial of Mesenchymal Stem Cells in COPD. Chest, 2013. 143(6): p. 1590-1598.
[9] Ribeiro-Paes, J. T., et al., Unicentric study of cell therapy in chronic obstructive pulmonary disease/pulmonary emphysema. International Journal of Chronic Obstructive Pulmonary Disease, 2011. 6: p. 63-71.
[10] Ribeiro-Paes, J. T., et al., A protocol proposition of cell therapy for the treatment of chronic obstructive pulmonary disease. Rev Port Pneumol, 2014. 20(2): p. 84-91.
[11] Stolk, J., et al., A phase I study for intravenous autologous mesenchymal stromal cell administration to patients with severe emphysema. QJM, 2016. 109(5): p. 331-6.
[12] Cheng, S.-L., C.-H. Lin, and C.-L. Yao, Mesenchymal Stem Cell Administration in Patients with Chronic Obstructive Pulmonary Disease: State of the Science. Stem Cells International, 2017. 2017: p. 1-14.
[13] Liu, X., Q. Fang, and H. Kim, Preclinical Studies of Mesenchymal Stem Cell (MSC) Administration in Chronic Obstructive Pulmonary Disease (COPD): A Systematic Review and Meta-Analysis. PLoS One, 2016. 11(6): p. e0157099.
[14] Antunes, M. A., et al., Mesenchymal stem cell trials for pulmonary diseases. J Cell Biochem, 2014. 115.
[15] Antunes, M. A., et al., Effects of different mesenchymal stromal cell sources and delivery routes in experimental emphysema. Respiratory Research, 2014. 15(1): p. 118.
[16] Francis, G. L., Albumin and mammalian cell culture: implications for biotechnology applications. Cytotechnology, 2010. 62(1): p. 1-16.
[17] Boldt, J., Use of albumin: an update. Br J Anaesth, 2010. 104(3): p. 276-84.
[18] Raoufinia, R., et al., Overview of Albumin and Its Purification Methods. Adv Pharm Bull, 2016. 6(4): p. 495-507.

What is claimed is:

1. A pharmaceutical composition for treating chronic obstructive pulmonary disease, comprising:
an effective amount of human mesenchymal stem cells;
human serum albumin; and
a pharmaceutically acceptable carrier or diluent.

2. The pharmaceutical composition of claim 1, wherein the human serum albumin is in an amount ranging from 0.5% (w/v) to 25% (w/v), based on the volume of the pharmaceutically acceptable carrier or diluent.

3. The pharmaceutical composition of claim 2, wherein the human serum albumin is in an amount ranging from 1% (w/v) to 10% (w/v), based on the volume of the pharmaceutically acceptable carrier or diluent.

4. The pharmaceutical composition of claim 1, which is prepared by a method comprising mixing the human mesenchymal stem cells with the pharmaceutically acceptable carrier or diluent, which is supplemented with the human serum albumin.

5. A method for treating chronic obstructive pulmonary disease in a subject in need thereof, comprising:
administering to the subject a pharmaceutical composition comprising: an effective amount of human mesenchymal stem cells; human serum albumin; and a pharmaceutically acceptable carrier or diluent.

6. The method of claim 5, wherein the human serum albumin is in an amount ranging from 0.5% (w/v) to 25% (w/v), based on the volume of the pharmaceutically acceptable carrier or diluent.

7. The method of claim 5, wherein the human serum albumin is in an amount ranging from 1% (w/v) to 10% (w/v), based on the volume of the pharmaceutically acceptable carrier or diluent.
